(19) United States
(12) Patent Application Publication
(10) Pub. No.: US 2022/0029071 A1
(43) Pub. Date: Jan. 27, 2022

(54) OPTOELECTRONIC DEVICE HAVING A LIGHT-EMITTING DIODE EMITTING IN THE ULTRAVIOLET ON WHICH AN OPTICAL DEVICE IS ARRANGED

(71) Applicant: COMMISSARIAT AL L'ENERGIE ATOMIQUE ET AUX ENERGIES ALTERNATIVES, Paris (FR)

(72) Inventors: David VAUFREY, Grenoble (FR); Yohan DESIERES, Grenoble (FR)

(73) Assignee: COMMISSARIAT A L'ENERGIE ATOMIQUE ET AUX ENERGIES ALTERNATIVES, Paris (FR)

(21) Appl. No.: 17/298,147

(22) PCT Filed: Nov. 26, 2019
(86) PCT No.: PCT/FR2019/052810

§ 371 Date: May 28, 2021

(51) Int. Cl. H01L 33/50; H01L 33/00; H01L 33/54; A61L 2/10; C09K 11/62

(52) U.S. Cl. CPC H01L 33/505; H01L 33/005; H01L 33/502; H01L 2933/005; A61L 2/10; C09K 11/62; H01L 2933/0041; H01L 33/54

(57) ABSTRACT

The optoelectronic device includes a light-emitting diode configured to emit an electromagnetic radiation at an emission wavelength of the light-emitting diode in the ultraviolet. The optoelectronic device includes an optical device configured to extract photons generated by the light-emitting diode, said optical device being arranged on an emission face of the light-emitting diode, said optical device including transparent particles at the emission wavelength of the light-emitting diode. The optical device includes conversion particles configured to emit, by converting a part of the electromagnetic radiation emitted by the light-emitting diode, an electromagnetic radiation at an emission wavelength of the conversion particles included in the ultraviolet and strictly greater than the emission wavelength of the light-emitting diode.

### TECHNICAL FIELD OF THE INVENTION

The technical field of the invention concerns that of optoelectronics and more particularly that of optoelectronic devices emitting in the ultraviolet. In particular, the invention concerns an optoelectronic device including a light-emitting diode configured to emit an electromagnetic radiation at an emission wavelength of the light-emitting diode comprised in the ultraviolet.

### STATE OF THE ART

It is known to use ultraviolet light, that is to say an ultraviolet electromagnetic radiation, to carry out disinfection. For this, it can be used at least one light-emitting diode having, during its operation, an emission spectrum having a peak whose wavelength is comprised in the ultraviolet. Of course, for such a light-emitting diode to be effective, we seek to improve its efficiency so that it is as high as possible, in particular by improving the extraction of photons that the light-emitting diode can generate.

To obtain a high efficiency light-emitting diode, it is known practice to limit the zo trapping of photons in the thin semiconductor layers of the light-emitting diode by total internal reflection. To avoid this phenomenon of total internal reflection, a first technique is known consisting in roughening the face, or surface, of emission of the light-emitting diode, and a second technique consisting in adding a dome of high optical index relative to the effective index of the light-emitting diode, for example epoxy dome, on the emission face of the light-emitting diode. These first and second techniques can be used alone or in combination. However, these first and second techniques are not suitable for light-emitting diodes emitting in the ultraviolet. This maladjustment stems in particular from:
that the structuring to roughen a semiconductor layer of a light-emitting diode emitting in the ultraviolet damages the electrical properties of this layer: that is to say that there is an increase in non-radiative recombination, and
that epoxy is highly absorbent at wavelengths comprised in the ultraviolet.
   This results in the disadvantage that the aforementioned first and second techniques are not suitable to be applied to a light-emitting diode emitting in the ultraviolet. There is therefore a need to find a solution allowing to improve the extraction of photons generated by a light-emitting diode emitting in the ultraviolet. The book «III-Nitride Ultraviolet Emitters: Technology and Applications» by Michael Kneissl and Jens Rass, Springer Series in Materials Science 227 describes in particular on pages 13 to 15 the problems linked to the extraction of photons from an optoelectronic device having a light-emitting diode emitting in the ultraviolet.

Moreover, it is known in the technical field of ultraviolet disinfection to use an ultraviolet C (UV-C) radiation combined with ultraviolet A (UV-A) radiation to improve the destruction of bacteria, for example present in the air or in water. The UV-C allows destroying the DNA (deoxyribonucleic acid) bonds of bacteria subjected to this UV-C. So it is possible to believe that just using UV-C would be enough to destroy bacteria. However, in practice, DNA repair methods may go so far as to counteract the effect of destroying DNA bonds, so that it may be necessary to achieve the desired disinfection effect to cause irreversible damage to bacteria. Thus, to obtain the desired disinfection effect, it is known to combine the effect of UV-C with that of UV-A as described in the document «Effect of coupled UV-A and UV-C LEDs on both microbiological and chemical pollution of urban wastewaters» by A.-C Chevremont et al. published in Science of the Total Environment 426 (2012), pages 304 to 310, by publisher Elsevier. Such a combination of the effects of UV-C and UV-A can be achieved by juxtaposing two light-emitting diodes with different emission wavelengths. There is a need to improve this solution which uses two separate light-emitting diodes. Indeed, the use of two light-emitting diodes to generate the UV-A and the UV-C has the particular drawback of requiring a power supply for each of the light-emitting diodes, thus complicating the implementation of the disinfection since this requires independent control of the two light-emitting diodes.

It is also known from the state of the art of the American patent application published under the number US 2017/0104138 A1, this patent application relating to an ultraviolet light-emitting device.

### OBJECT OF THE INVENTION

The aim of the invention is to allow to improve the extraction of photons generated within an optoelectronic device, in particular generated by a light-emitting diode having an emission wavelength comprised in the ultraviolet. In particular, the invention seeks to improve disinfection by using a light-emitting diode emitting in the ultraviolet.

To this end, the invention relates to an optoelectronic device including a light-emitting diode configured to emit an electromagnetic radiation at an emission wavelength of the light-emitting diode comprised in the ultraviolet, this optoelectronic device being characterized in that it includes an optical device configured to extract photons generated by the light-emitting diode, said optical device being arranged on an emission face of the light-emitting diode, said optical device including particles transparent at the emission wavelength of the light-emitting diode, preferably said transparent particles are made of a semiconductor material.

Such an optoelectronic device allows to tend towards the desired aim in the sense that its optical device is, at least in part, transparent to the ultraviolet radiation emitted by the light-emitting diode, and in the sense that the transparent particles allow to give the desired shape to the optical device, for example to form a rough aspect surface, or even to form an optical element for extracting photons and shaping a beam of photons to be emitted by the optoelectronic device. Another advantage of transparent particles is that they can serve, as will be seen below, as a matrix in which are dispersed so-called conversion particles allowing to convert a part of the electromagnetic radiation emitted by the light-emitting diode during its operation. Yet another advantage of transparent particles is that the cohesion of the optical device can be maintained by van der Waals forces implemented, for example, between particles whether they are transparent or not: the manufacture of such an optoelectronic device can therefore be implemented without the need for annealing which could damage the layers present within the light-emitting diode, the optoelectronic device can thus have satisfactory photon extraction efficiency.

The optoelectronic device may further include one or more of the following characteristics:
the optical device includes conversion particles configured so as to emit, by converting a part of the electromagnetic radiation emitted by the light-emitting diode, an electromagnetic radiation at an emission wavelength of the conversion particles comprised in the ultraviolet and strictly greater than the emission wavelength of the light-emitting diode;
the emission wavelength of the light-emitting diode is selected in ultraviolet C, and the emission wavelength of the conversion particles is selected in ultraviolet A;
the optical device has the shape of a dome, a cone or a pyramid;
the optical device includes a plurality of structures, these structures being arranged on the emission face of the light-emitting diode, each of the structures including a part of the transparent particles;
the structures each include a part of the conversion particles;
the optical device includes an optical element and structures, the structures being arranged on the emission face of the light-emitting diode, the optical element being arranged on the structures, the optical element includes the conversion particles, the transparent particles being distributed such that: the structures each include transparent particles, and the optical element includes transparent particles;
the optical device includes an optical element and structures, the structures being arranged on the emission face of the light-emitting diode, the optical element being arranged on the structures, and the conversion particles are distributed so that the structures each include conversion particles, the transparent particles being distributed so that the optical element includes transparent particles and that the structures include transparent particles;
the optical device includes an optical element and structures, the structures being arranged on the emission face of the light-emitting diode, the optical element being arranged on the structures, the structures including only the conversion particles and the optical element including transparent particles;
the transparent particles are each made of aluminum nitride;
the conversion particles are each made of gallium nitride;
each of the transparent particles has a size strictly less than λ/(2×n), with n the optical index of the material of the transparent particles, and A the emission wavelength of the light-emitting diode;
each of the conversion particles has a size strictly less than λ/(2×n₁), with n₁ the optical index of the material of the conversion particles, and A the emission wavelength of the light-emitting diode;
the cohesion of the optical device is ensured by van der Waals bonds.

The invention also relates to a method for manufacturing an optoelectronic device, in particular as described. Such a manufacturing method includes a step of supplying the light-emitting diode and a step of forming the optical device. The step of forming the optical device includes a step of depositing a solution at least above the emission face of the light-emitting diode, said solution including a solvent and at least a part of the transparent particles. The step of forming optical device includes a step of evaporating the solvent from the deposited solution to form at least a part of the optical device.

According to a particular embodiment of the manufacturing method, the solution deposited at least above the emission face is a first solution including a first part of the transparent particles of the optical device to be formed. The step of forming the optical device includes a step of shaping the first deposited solution. It results from the step of shaping the first deposited solution and the step of evaporating the solvent from the first deposited solution, a formation of a first part of the optical device including structures. The step of forming the optical device includes a step of depositing a second solution on the structures in order to form a second part of the optical device, the second solution including a solvent and a second part of the transparent particles of the optical device to be formed. The step of forming the optical device includes a step of evaporating the solvent from the second deposited solution to form the second part of the optical device. According to this particular embodiment, the first solution, or the second solution, includes conversion particles configured so as to emit, by converting a part of the electromagnetic radiation emitted by the light-emitting diode, an electromagnetic radiation at an emission wavelength of the conversion particles comprised in the ultraviolet and strictly greater than the emission wavelength of the light-emitting diode.

### BRIEF DESCRIPTION OF THE DRAWINGS

The invention will be better understood on reading the detailed description which follows, given only by way of non-limiting example and made with reference to the accompanying drawings and listed below.

FIG. 1 illustrates a cross-sectional view of an optoelectronic device according to a particular embodiment of the invention.

FIG. 2 illustrates a cross-sectional view of the optoelectronic device according to another particular embodiment of the invention.

FIG. 3 illustrates a cross-sectional view of the optoelectronic device according to yet another particular embodiment of the invention.

FIG. 4 illustrates, in section view, a portion of an optical device of the optoelectronic device showing that, according to one embodiment, the latter includes an assembly of transparent particles.

FIG. 5 illustrates, in section view, a portion of the optical device of the optoelectronic device showing that, according to one embodiment, the latter includes an assembly of transparent particles and of conversion particles.

FIG. 6 illustrates a step of a manufacturing method of the optoelectronic device.

FIG. 7 illustrates a step of the manufacturing method carried out in order to form structures of the optical device.

FIG. 8 illustrates a step of the manufacturing method allowing forming the structures of the optical device after the step represented in FIG. 7.

FIG. 9 illustrates a step of the manufacturing method carried out in order to form the optoelectronic device represented in FIG. 1.

FIG. 10 illustrates, according to an alternative to FIG. 9, a step of the manufacturing method carried out in order to form the optoelectronic device represented in FIG. 1.

FIG. 11 illustrates a step of the manufacturing method implemented after that represented in FIG. 10.

FIG. 12 illustrates a step of the manufacturing method carried out in order to form the optoelectronic device represented in FIG. 3.

FIG. 13 illustrates, according to an alternative to FIG. 12, a step of the manufacturing method carried out in order to form the optoelectronic device represented in FIG. 3.

In these figures, the same references are used to designate the same elements.

Moreover, the elements represented in the figures are not necessarily to scale in order to facilitate the understanding of these figures.

### DETAILED DESCRIPTION

In the present description, when it is mentioned «an electromagnetic radiation at a wavelength», it is meant that this wavelength is that of the peak of the emission spectrum of this electromagnetic radiation. The peak of the emission spectrum therefore corresponds to a wavelength value at which the most part of the electromagnetic radiation concerned is emitted. Moreover, when referring to «an emission wavelength» of an emissive system such as a light-emitting diode, or conversion particles, it is a question of a wavelength at which an electromagnetic radiation can be emitted by this emissive system. This electromagnetic radiation has, when emitted by the emissive system, a wavelength equal to the emission wavelength.

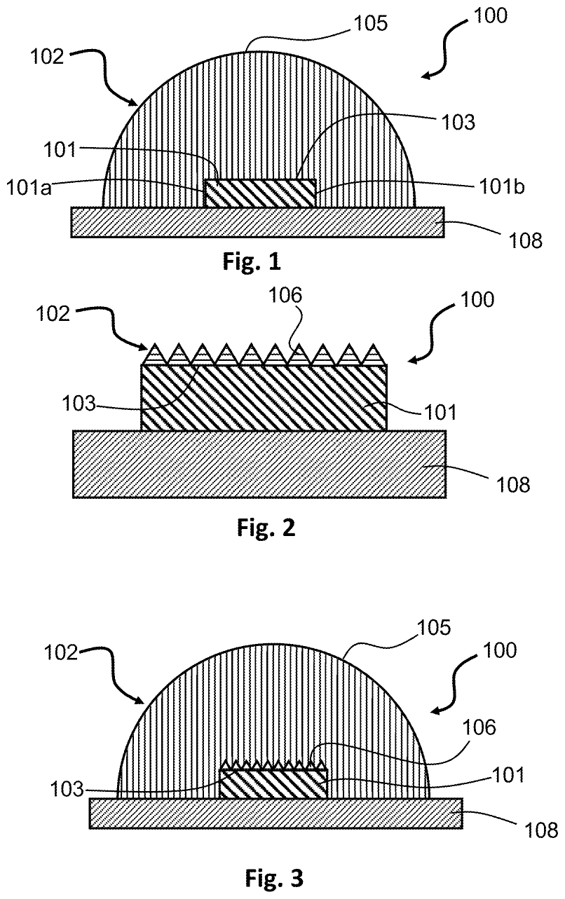

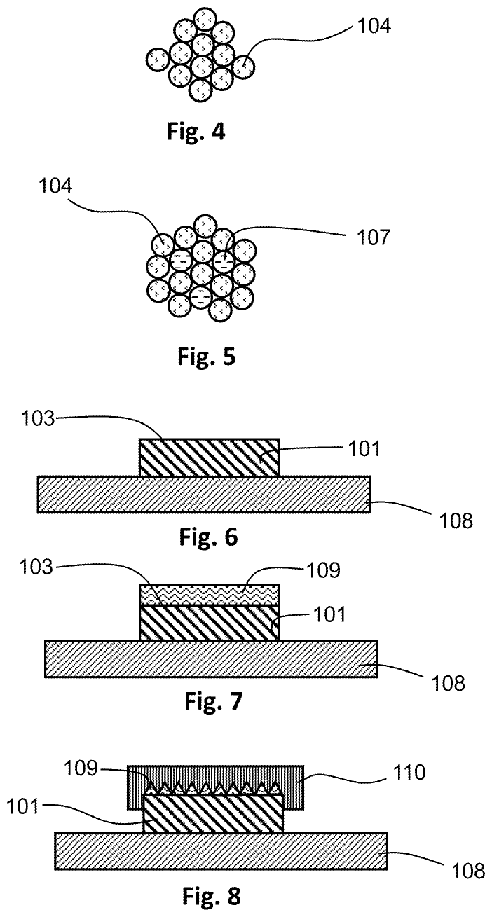

As illustrated by way of example in FIGS. 1 to 3, the invention relates to an optoelectronic device 100 including a light-emitting diode 101 configured to emit an electromagnetic radiation according to an emission wavelength of the light-emitting diode 101 comprised in ultraviolet, preferably comprised between 100 nm and 400 nm. Furthermore, the optoelectronic device 100 includes an optical device 102 for extracting photons generated by the light-emitting diode 101, in other words, the optical device 102 is configured to extract photons generated by the light-emitting diode 101 efficiently. Where appropriate, as will be described below, the optical device 102 can also ensure a frequency conversion to allow the generation of a radiation of lower optical frequency than the radiation emitted by the light-emitting diode 101. This optical device 102 is arranged on an emission face 103 of the light-emitting diode 101. More precisely, the optical device 102 is in contact with the emission face 103 to ensure the extraction of photons generated by the light-emitting diode 101 during its operation. The emission face 103 of the light-emitting diode 101, also called the emissive face, corresponds in particular to a face by which the passage of a majority of the photons escaping from the light-emitting diode 101 and generated by the light-emitting diode 101 is desired, in particular in an active region of the latter, during its operation. The optical device 102 is therefore configured to exacerbate the extraction of photons generated by the light-emitting diode 101 during its operation. The optical device 102 can also allow, where appropriate, to cooperate with the light-emitting diode 101 to shape a beam of photons with or without conversion of these photons, as will be seen below. The optical device 102 further includes particles 104 transparent (FIGS. 4 and 5) to the electromagnetic radiation emitted by the light-emitting diode 101, these transparent particles 104 participate in particular in improving the extraction of photons generated by the light-emitting diode towards the light-emitting diode outwards of the optoelectronic device 100. By «particles 104 transparent to the electromagnetic radiation emitted by the light-emitting diode 101», it is in particular understood that these transparent particles 104 are transparent at the emission wavelength of the light-emitting diode 101. This transparency of the transparent particles 104 is necessary to allow at least a part of the photons generated by the light-emitting diode 101 to pass through the optical device 102 unlike the epoxy of the prior art which is not transparent to ultraviolet. The transparent particles 104 are made of a transparent material such as, for example, a semiconductor material, in particular with a bandgap energy strictly greater than the energy of the photons emitted by the light-emitting diode 101. Such transparent particles 104 exhibit the advantage of improving the extraction of photons from electromagnetic radiation, of the ultraviolet type, emitted by the light-emitting diode 101, without in particular having to form a bulk layer in this material whose deposition zo and any structuring would be liable to damage the light-emitting diode 101 which could then have a degraded efficiency. Moreover, the use of transparent particles 104 allows to structure the optical device 102 in an appropriate manner without requiring technological steps carried out at a temperature strictly above 200° C. which could damage the light-emitting diode 101: which result in that the optoelectronic device 100 exhibits good efficiency. The transparent particles 104 can be used to form, for example, at least in part an optical element 105 (FIGS. 1 and 3), for example taking the form of a dome, on the light-emitting diode 101 and/or at least in part structures 106 (FIGS. 2 and 3) on the emission face 103 of the light-emitting diode 101. In FIG. 1, the optical element 105 consist of the optical device 102. In FIG. 2, the structures 106 consist of the optical device 102. In FIG. 3, the optical device 102 includes the structures 106 and the optical element 105. This optical element 105 and/or these structures 106 allow to prevent the photons generated within the optoelectronic device 100 from being reflected towards the inside of the light-emitting diode 101. To avoid this internal reflection, the transparent particles 104 in contact with the light-emitting diode 101 are in particular made of a material with an optical index greater than or equal to the effective index of the light-emitting diode 101. In particular, the optical device 102 includes an outer surface adapted to prevent a photon from being in a condition of total reflection. For example, the dome shape of the optical element 105 allows a photon, whatever its incidence, to encounter a diopter almost normal to its direction of propagation to avoid its internal reflection within the optical element 105. The optical element 105 is also preferably configured to shape a beam to be emitted by the optoelectronic device 100, this beam comprising photons generated by the optoelectronic device 100 for example by the light-emitting diode 101, or by the light-emitting diode 101 and in the optical device 102.

To allow the desired transparency, the semiconductor material forming the transparent particles 104 may be such that it has a gap, also called a bandgap in the field of microelectronics, strictly greater than the energy of the photons of the electromagnetic radiation emitted by the light-emitting diode 101. This energy of the photons emitted/generated by the light-emitting diode 101 is that of the ultraviolet photons. The advantage of using a semiconductor material allows, according to its choice, to let photons generated by the light-emitting diode 101 to pass. Thus, the semiconductor material of the transparent particles 104 has the advantage of ensuring the desired transparency at the emission wavelength of the light-emitting diode 101. Furthermore, an advantage of using transparent particles 104 of semiconductor material is that such particles can be deposited during the manufacture of the optoelectronic device 100 according to methods that are simple to implement such as, for example, centrifugation or the deposition of a drop: in this sense, the described optoelectronic device 100 can easily be manufactured, in particular without having the drawback of damaging the light-emitting diode 101 during the formation of the optical device 102.

Moreover, the material, in particular semiconductor, of the transparent particles 104 preferably has an optical index (also called refractive index) greater than or equal to the effective index of the light-emitting diode 101 to improve the extraction of the photons generated by the light-emitting diode 101. The effective index of the light-emitting diode corresponds to the optical index seen by the optical mode propagating in the light-emitting diode 101: thus, the effective index of the light-emitting diode 101 is comprised between the smallest and the largest of the optical indices of the semiconductor layers of the light-emitting diode 101. Moreover, considering that the optical device 102 has an effective index corresponding to the optical index seen by the optical mode propagating in the optical device 102 (comprised especially between the largest and the smallest of the optical indices of what makes up the optical device 102), this effective index of the optical device 102 is in particular greater than or equal to the effective index of the light-emitting diode 101 in order to ensure proper extraction of photons generated by the light-emitting diode 101.

According to a particular embodiment, the transparent particles 104 each include aluminum nitride (for example AlN), and are preferably each made of aluminum nitride. Each transparent particle 104 can have a size of 5 nm, and can have a density of 3.26 g/cm³. These characteristics of the transparent particles 104, taken individually or in combination, are very particularly suitable in the context of the optical device 102 as described, in particular for shaping the optical device 102 in any suitable shape. Aluminum nitride is very particularly suitable when the emission wavelength of the light-emitting diode 101 is in the ultraviolet, in particular comprised in the UV-C (ultraviolet C), but also in the ultraviolet B (UV-B) or in ultraviolet A (UV-A). Alternatively, the transparent particles 104 may include, or be made of, aluminum oxide (for example of formula Al₂O₃), however aluminum nitride is preferred because its optical index is higher than that of aluminum oxide.

The transparent particles 104 can participate in directly forming the optical device 102 or a part of the latter whose roughness (for example of an amplitude strictly greater than 1/10 of the wavelength of the radiation emitted in the light-emitting diode 101) allows to exacerbate the extraction of photons while avoiding implementing a modification of the surface of a semiconductor layer of the light-emitting diode 101 subsequent to its deposition, which would have the consequence of damaging its electrical properties.

It was mentioned in the part relating to the state of the art that, for a disinfection application seeking to destroy bacteria, it could be advantageous to emit ultraviolet radiation at two different wavelengths. There is therefore a need to find a solution allowing such a transmission. To meet this need, it could be considered to couple a UV-A diode and a UV-C diode as taught in the document «Effect of coupled UV-A and UV-C LEDs on both microbiological and chemical pollution of urban wastewaters» By A.-C Chevremont et al. published by the publisher Elsevier in Science of the Total Environment 426 (2012) pages 304 to 310. However, such a solution requires the presence of two separate light-emitting diodes, therefore two power supply systems for these light-emitting diodes. To avoid having to resort to two separate diodes to carry out the emission of UV-A and UV-C, a preferred solution is to integrate conversion particles 107 to the optical device 102 (visible in FIG. 5). Thus, the optical device 102 may include conversion particles 107 configured so as to emit, by converting a part of the electromagnetic radiation emitted by the light-emitting diode 101, an electromagnetic radiation at an emission wavelength of the conversion particles 107 comprised in the ultraviolet and strictly greater than the emission wavelength of the light-emitting diode 101. In other words, the electromagnetic radiation emitted by the conversion particles 107 during the operation of the light-emitting diode 101 has a wavelength comprised in the ultraviolet and strictly greater than the wavelength of the electromagnetic radiation emitted by the light-emitting diode 101. Thus, it is possible to convert a part of the photons emitted by the light-emitting diode 101 during its operation. The use of conversion particles 107 allows the use of only a single light-emitting diode 101 which, in conjunction with optical device 102, enables optoelectronic device 100 to emit two electromagnetic radiations with distinct emission peaks. Thus, the beam emitted by the optoelectronic device 100 can include photons of two electromagnetic radiations having different wavelengths.

Preferably, the emission wavelength of the light-emitting diode 101 is selected from UV-C, and the emission wavelength of the conversion particles 107 is selected from UV-A. Thus, it is possible, on the one hand, to destroy DNA bonds (effect of UV-C) in bacteria, and on the other hand to destroy the membrane of bacteria cells containing the DNA to be destroyed (effect of UV-A) to prevent DNA from rebuilding. Preferably, to achieve this double destruction, the emission wavelength of the light-emitting diode 101 is comprised between 230 nm and 300 nm, in particular the emission wavelength of the light-emitting diode 101 is equal to 265 nm. Preferably, to achieve this double destruction, the emission wavelength of the conversion particles 107 is comprised between 300 nm and 400 nm, in particular the emission wavelength of the conversion particles 107 is equal to 365 nm.

The conversion particles 107 allow in particular, by optical pumping, a conversion to a wavelength strictly greater than the emission wavelength of the light-emitting diode 101 when they receive the electromagnetic radiation emitted by the light-emitting diode 101. Thus, such an optoelectronic device 100 has the advantage of forming two sources capable of emitting respectively two electromagnetic radiations of different wavelengths thanks to a single electrical supply supplying the light-emitting diode 101. The light-emitting diode 101 forms one of these two sources and the conversion particles 107 form the other of these two sources. The photon fluxes resulting from the two electromagnetic radiations can be calibrated as a function of the used light-emitting diode 101 type, and of the composition of transparent particles 104 and of conversion particles 107 of the optical device 102.

The conversion particles 107 may each include, or are each made of, gallium nitride (for example of formula GaN). Such conversion particles 107 are most particularly suitable for emitting ultraviolet radiation with a wavelength greater than that of the ultraviolet radiation having excited said conversion particles 107 as described in the document «Simple synthesis of GaN nanoparticles from gallium nitrate and ammonia aqueous solution under a flux of ammonia gas» by Ferry Iskandar et al. published in Materials Letters 60 (2006) 73-76 by publisher Elsevier. More generally, the conversion particles 107 can each be formed from a semiconductor material such that it has a gap strictly less than the energy of the photons of the electromagnetic radiation emitted by the light-emitting diode 101, and corresponding to the energy of the photons to be emitted by the conversion particles 107. According to a preferred embodiment allowing to implement the conversion of UV-C into UV-A, the transparent particles 104 are each made of aluminum nitride and the conversion particles 107 are each made of gallium nitride. Alternatively, the transparent particles 104 can each be made of aluminum oxide.

An advantage of the optical device 102 including the transparent particles 104 and the conversion particles 107 is that the conversion particles 107 can be dispersed within a set, also called a matrix, of transparent particles 104 as described.

Thus, the transparent particles 104 allow, during the manufacture of the optoelectronic device 100, to control the number of conversion particles 107 and the distribution of the conversion particles 107 within the optical device 102. This allows to adjust the proportion of photons, of the electromagnetic radiation emitted by the light-emitting diode 101 during its operation, transformed into photons of the electromagnetic radiation emitted by the conversion particles 107. Of course, knowing this, one skilled in the art is able to adapt:
the quantity and the dimensions of the conversion particles 107 within the optical device 102,
the quantity and the dimensions of the transparent particles 104 within the optical device 102,
the dimensions of the optical device 102,
   the whole according to the flux of UV-A photons and the flux of UV-C photons desired to be emitted by the optoelectronic device 100.

In particular, the conversion particles 107 can each have a size comprised between 5 nm and 100 nm. This size is very particularly suitable for the application for converting the radiation emitted by the light-emitting diode 101. This size range for the conversion particles 107 is very particularly suitable for allowing to control, where appropriate, the mixing of these conversion particles with the transparent particles, in particular when the transparent particles 104 are similar in size to the size of the conversion particles 107.

In the present description, the size of a (transparent or conversion) particle is in particular its maximum dimension. In particular, the particle has a size such that it is included in a sphere whose diameter is equal to this size. Preferably, each particle referred to in the present description adopts the shape of a sphere. The described particles are in particular nanoparticles which can adopt the shape of beads.

To allow the optoelectronic device 100 to emit a flux of photons generated by the conversion particles 107, the transparent particles 104 are also transparent to the electromagnetic radiation emitted by the conversion particles 107. Thus, where appropriate, the semiconductor material forming these transparent particles 104 is such that it has a gap strictly greater than the energy of the photons of the electromagnetic radiation emitted by the conversion particles 107 during the operation of the light-emitting diode 101.

It is known that to exacerbate the extraction of photons, it is possible to use an encapsulant suitable for the light-emitting diode, or to roughen a surface of a layer in which the photons is displaced. The use of transparent particles 104, or of transparent particles 104 and of conversion particles 107, allows this in the particular case of a light-emitting diode 101 emitting in the ultraviolet: it is then sufficient to arrange the particles to obtain a suitable shape and/or a suitable composition of the optical device 102. In the context of the conversion by the conversion particles 107, the porosity of the optical device 102 (conferred by the presence of interstices between the particles), the shape and the roughness of surface of the optical device 102 conferred by the assembly of the particles will allow to reduce the trapping of the photons coming from the conversion particles 107. In particular, FIGS. 1 to 3 illustrate different possibilities for making the optoelectronic device 100. Where appropriate, the optical device 102 allows exacerbating:
the extraction of photons emitted by the light-emitting diode 101 to the outside of the optoelectronic device 100, or
the extraction of photons emitted by the light-emitting diode 101 and by the conversion particles 107 to the outside of the optoelectronic device 100.

According to an embodiment, in particular illustrated in FIG. 1, the optical device 102 allows to transmit the electromagnetic radiation emitted by the light-emitting diode 101, in particular by converting a part of this electromagnetic radiation emitted by the light-emitting diode 101 in the event of the presence of conversion particles 107 in the optical device 102 also called optical element 105 according to this embodiment. To provide this transmission function, the optical device 102, or the optical element 105, may have the shape of a dome 105, a pyramid or a cone. In particular, the dome can be spherical and take the shape of a hemisphere. In particular, the pyramid may have a base formed by a polygon, for example a triangle, a square or a hexagon. Other shapes of the base of the pyramid can be considered. The pyramid can be truncated. Here, the shape is in particular a so-called «outer» shape of the optical device 102 conferred by its outer surface remote from the light-emitting diode 101. Preferably, the optical device 102 encapsulates a part of the light-emitting diode 101. Preferably, this optical device 102 includes, or consists of, the transparent particles 104 with electromagnetic radiation emitted by the light-emitting diode 101. In particular, the transparent particles 104 are made secured to one another by van der Waals bonds. The optical device 102, in particular in the form of a dome, can optimize the power transmission, that is to say the extraction of photons, from its outer surface, in particular by virtue of the assembly of the particles which compose it and which can allow to increase the number of facets of the optical device 102 and/or the roughness of its outer surface to improve the extraction of photons. For example, for a dome, its outer surface corresponds to its rounded surface opposite to the light-emitting diode 101. Furthermore, such an optical device 102 also allows to participate in the shaping of the electromagnetic radiation emitted by the light-emitting diode 101 and passing through the optical device 102 according to a desired beam of photons. The optical device 102, for example in the form of a dome 105, can cover the light-emitting diode 101, in particular so as to facilitate the extraction of photons from side faces 101a, 101b (FIG. 1) of the light-emitting diode 101 distinct from its emission face 103: it is then possible to maximize the quantity of photons extracted from the optoelectronic device 100. Here, the optical device 102 can include only the transparent particles 104, or where appropriate the transparent particles 104 and the conversion particles 107.

According to another embodiment, as for example illustrated in FIG. 2, the optical device 102 includes a plurality of structures 106. These structures 106 are arranged on the emission face 103 of the light-emitting diode 101. Each of these structures 106 includes a part of transparent particles 104. In other words, the transparent particles 104 of the optical device 102 are distributed within the different structures 106. These structures 106 can be micrometric pyramids in particular consisting of, or including the, transparent particles 104. The top of each of the pyramids is at a distance from the light-emitting diode 101 while the base of each of the pyramids may be in contact with the light-emitting diode 101. The term «micrometric pyramids» means that these pyramids have dimensions in particular comprised between 0.5 μm and 10 μm. In fact, the structures 106 can be arranged so as to form a layer of structures 106 joining together on the emission face 103. Such structures 106 are configured to improve the light extraction (ultraviolet light) of the light-emitting diode 101 in particular by facilitating the passage of photons emitted by the light-emitting diode 101 through the layer of structures 106 while limiting the return of photons by internal reflection to the light-emitting diode 101. Any form of structure 106 allowing the extraction function referred to above can be used. In the event that the conversion is not desired, the structures 106 consist of the transparent particles 104. In the case where the conversion of a part of the electromagnetic radiation emitted by the light-emitting diode is desired, the structures 106 each include a part of the transparent particles 104 and a part of the conversion particles 107 (the conversion particles 107 are then distributed in structures 106). The structures 106 are preferably identical. The formation of a plurality of structures here allows to increase the number of facets of the optical device 102 to improve the extraction of photons to the exterior of the optoelectronic device 100.

In FIG. 3, the optical device 102 is a combination of the structures 106, in particular as described above, with an optical element 105, in particular as described above. In other words, the optical device 102 may include the optical element 105 and the structures 106, the structures 106 being arranged on the emission face 103 of the light-emitting diode 101, the optical element 105 being arranged on the structures 106. Thus, it will be understood that in FIG. 3, the optical device 102 includes two parts, one formed by the structures 106, and the other formed by the optical element 105, in particular one of these parts includes both a first part of the transparent particles 104 and the conversion particles 107 and the other of these parts includes a second part of the transparent particles 104. In particular, the structures 106 have the advantage here of promoting at least the passage of photons generated by the light-emitting diode 101 towards the optical element 105, and where appropriate the passage of photons generated by the conversion particles 107 present in the structures 106 towards the optical element 105. Furthermore, the optical element 105 allows in particular to shape a beam of photons emitted by the optoelectronic device 100, this beam of photons comprising photons of the electromagnetic radiation emitted by the light-emitting diode 101 and photons of the electromagnetic radiation emitted by the conversion particles 107. Moreover, the outer surface of the optical element 105 delimited by portions of particles (in particular transparent, or transparent and conversion particles) also participates in improving the extraction of the photons generated within the optoelectronic device.

According to a particular embodiment of the combination of the optical element 105 with the structures 106, the optical element 105 includes the conversion particles 107 and the transparent particles 104 are distributed such that the structures 106 each include transparent particles 104 and that the optical element 105 includes transparent particles 104. In other words, the transparent particles 104 are distributed in the structures 106 and in the optical element 105. Preferably, the structures 106 here only include transparent particles 104. Such a distribution conversion particles 107 and transparent particles 104 is advantageous in the sense that the volume of the optical element 105, strictly greater than the volume of each of the structures 106, allows to more easily control, because of this larger volume, the proportion of conversion particles 107 in the optical element 105 compared to the case where these conversion particles 107 would be placed in structures 106.

According to another particular embodiment of the combination of the optical element 105 with the structures 106, the conversion particles 107 are distributed so that the structures 106 each include conversion particles 107. The transparent particles 104 are then distributed so that the optical element 105 includes transparent particles 104 and that the structures 106 include transparent particles 104. In other words, the transparent particles 104 are distributed in the structures 106 and in the optical element 105. Preferably, the optical element 105 includes here only transparent particles 104. This embodiment allows better coupling between the radiation emitted by the light-emitting diode 101 and the conversion particles 107 contained in the structures formed in contact with the emission face 103: thus the efficiency of the optical device 102 is improved.

According to yet another particular embodiment of the combination of the optical element 105 with the structures 106, the structures 106 include only the conversion particles 107 and the optical element 105 includes the transparent particles 104. This embodiment is particularly suitable for producing an efficient conversion.

According to the combination of the structures 106 with the optical element 105, the shape, in particular called the «outer shape» of the optical device 102 may be as described above. Particularly, the optical device 102, in particular the optical element 105, may have the shape of a dome, a pyramid, or a cone.

Generally, applicable in particular to the various embodiments illustrated in FIGS. 1 to 5, the cohesion of the optical device 102 is provided by van der Waals bonds. The term «cohesion of the optical device 102» means that it behaves like a component whose elements (here the particles) are attached to each other by van der Waals bonds. In other words, the transparent particles 104, or the transparent particles 104 and the conversion particles 107 are linked together by van der Waals bonds. More particularly for any pair of particles in contact, a van der Waals bond is formed between these particles of the pair, whether these particles of the pair are two transparent particles 104, two conversion particles 107, or one conversion particle 107 and one transparent particle 104. In particular, the optical device 102 consists of transparent particles 104 and of conversion particles 107 in particular linked together by van der Waals bonds. The advantage of having an optical device 102 whose cohesion is provided by van der Waals bonds is that it is not necessary to implement annealing at a high temperature which risks damaging the light-emitting diode 101 to form this optical device 102.

Preferably, each of the transparent particles 104 has a size strictly less than λ/(2×n), with n the optical index of the material of the transparent particles 104, and λ the emission wavelength of the light-emitting diode 101. This has the advantage of improving the extraction of the photons generated by the light-emitting diode 101 and, where appropriate, of the photons generated by the conversion particles 107 towards the outside of the optoelectronic device 100, in particular in contact between the outer surface of the optical device 102 with air.

Preferably, each of the conversion particles 107 has a size strictly less than λ/(2×n₁), with n₁ the optical index of the material of the conversion particles 107, and λ the emission wavelength of the light-emitting diode 101. This allows to improve the absorption, by said conversion particle 107, of photons generated by light-emitting diode 101.

The conditions given above relative to the size of the conversion or transparent particles as a function of the emission wavelength of the diode allow all of the particles present in the optical device 102 to behave as a homogeneous medium for the propagation of photons.

The optical device 102 can cooperate with any type of light-emitting diode 101 capable of emitting in the ultraviolet (in particular in the UV-C) by one or more emission faces, each emission face then being in contact with the optical device as described which in particular covers the light-emitting diode 101. For example, the light-emitting diode may be based on aluminum and gallium nitride.

It will be understood from what has been described above that when the optical device 102 includes conversion particles 107, it also allows to exacerbate the extraction of photons generated by these conversion particles 107 towards the outside of the optical device 102, in particular in a direction away from the light-emitting diode 101.

The optical device 102 may include interstices, in particular filled with air, formed between the (in particular transparent and, where appropriate, conversion) particles which compose it. These interstices are in particular present in the structures 106 and/or in the optical element 105. Preferably, an attempt is made to limit the presence of these interstices by choosing a suitable size of the particles in order to avoid excessively reducing the effective index of the optical device 102. In the case where the optical device 102 includes the transparent particles 104 and the conversion particles 107, the presence of interstices, in particular filled with air, between the particles, whether they are conversion or transparent, allows to decrease the effective index of the optical device 102 and to generate optical scattering to reduce the trapping of the radiation emitted by the conversion particles 107.

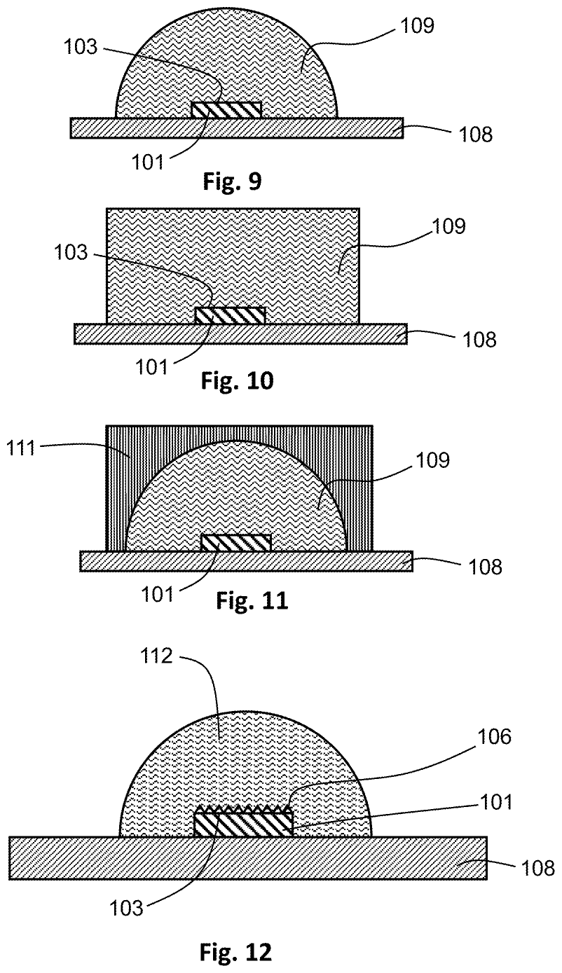

The invention also relates to a method for manufacturing an optoelectronic device 100, in particular as described. Particularly, the manufacturing method includes a step of supplying the light-emitting diode 101 (FIG. 6), for example formed on a substrate 108 (also represented in FIGS. 1 to 3 and 7 to 13), in particular a semiconductor substrate 108. This light-emitting diode 101 may have been manufactured beforehand by conventional techniques of microelectronics. Furthermore, the manufacturing method includes a step of forming the optical device 102 including a step of depositing a solution 109 (FIGS. 7, 9 and 10) at least above, or at least on, the emission face 103 of the light-emitting diode 101 provided for example according to FIG. 6. This solution 109 includes a solvent (for example water or ethanol) and at least a part of the transparent particles 104. If appropriate, the solution 109 deposited may also include the conversion particles 107. Then, the step of forming the optical device 102 may include a step of evaporating the solvent from the solution 109 deposited to form at least a part of the optical device 102. For example, the evaporation of the solvent from the solution 109 deposited in FIG. 9 allows to obtain the optical device 102 in the form of a dome as represented in FIG. 1. For example, the evaporation of the solvent from the solution 109 deposited in FIG. 7, after its shaping (FIG. 8), allows to obtain the structures 106 as visible in FIG. 2. For example, the evaporation of the solvent from the solution 109 deposited in FIG. 10, after its shaping (FIG. 11), allows to obtain the optical device 102 in the form of a dome as represented in FIG. 1. Generally, the evaporation of the solvent from the deposited solution 109 is advantageous because it does not require the use of temperatures liable to damage the light-emitting diode 101, the step of evaporating the solvent from the deposited solution 109 is preferably carried out at ambient temperature, or at 100° C. in order to limit the evaporation time. More generally, the step of evaporating the solvent from the deposited solution 109 is carried out at a temperature between 25° C. and 100° C. to avoid the deterioration of the light-emitting diode 101. The use of the solution 109 including transparent particles 104, and possibly conversion particles 107, allows to obtain either the optical device 102 or a part of the latter with a suitable distribution of the particles that it includes.

According to a first embodiment of this manufacturing method, the solution 109 deposited on the emission face 103 of the light-emitting diode 101 allows to form the optical device 102 in particular in its entirety.

For example, according to this first embodiment of the manufacturing method, the step of depositing the solution 109 is implemented by depositing a drop of the solution 109 (FIG. 9), in particular on the substrate 108 and on the emission face 103, whose drying by evaporation of the solvent which it contains allows to obtain the optical device 102 such as for example represented in FIG. 1. Here the advantage is that the solution 109 deposited directly has a shape representative of the desired shape of the optical device 102. In the event of depositing a drop, its viscosity is adapted so that its drying allows to obtain the optical device 102.

According to another example of this first embodiment, the solution 109 deposited (on the emission face 103 in FIG. 7 and on the emission face 103 and the substrate 108 in FIG. 10), for example by centrifugation, will be shaped (FIGS. 8 and 11) before evaporating the solvent. Here, after depositing the solution 109, the step of forming the optical device 102 may include a step of shaping the deposited solution 109 in order to form the optical device 102. The step of evaporating the solvent from the deposited solution 109 is in particular produced while the shape imparted to the solution 109 deposited by the shaping step is maintained throughout the step of evaporating the solvent from the deposited solution 109. This can be achieved through the use of a mold 110, 111 for shaping the deposited solution 109. The relative position of the mold 110, 111 with respect to the light-emitting diode 101 is maintained during the step of evaporating the solvent from the deposited solution 109 as illustrated in FIGS. 8 and 11. This technique is also known under the name of micro-stamping (or nano-printing). For example, the mold 110 of FIG. 8 allows to form the structures 106, and the mold 111 of FIG. 11 allows to form a dome as an optical device 102. According to this other example, at the end of the step of evaporating the solvent from the deposited solution 109, the mold 110, 111 is removed to obtain the optoelectronic device 100 either from FIG. 2 or from FIG. 1.

According to this first embodiment of the manufacturing method, the solution 109 can include the transparent particles 104, or the transparent particles 104 and the conversion particles 107.

According to a second embodiment of this manufacturing method, the solution 109 deposited on (or above) the emission face 103 of the light-emitting diode 101 allows to form a part of the optical device 102. It is then understood that step of forming the optical device 102 requires additional steps to be finalized.

Thus, according to the second embodiment of the manufacturing method, the optical device 102 can be in two parts. In this case, the solution 109 deposited (FIGS. 7 and 8) allows to form only a part of the optical device 102 called first part of the optical device 102. According to this second embodiment, the solution 109 deposited at least above, or the where appropriate on or at least on, the emission face 103 is a first solution including a first part of the transparent particles 104 of the optical device 102 to be formed. The manufacturing method, particularly the step of forming the optical device 102, also includes a step of shaping the first deposited solution 109 (FIG. 8) in order to form the first part of the optical device 102. The step of evaporating the solvent from the first deposited solution 109 is in particular carried out while the shape imparted to the first solution 109 by the shaping step is maintained, for example using a suitable mold 110 (FIG. 8). Moreover, it results from the step of shaping the first deposited solution 109 and the step of evaporating the solvent from the first deposited solution 109, a formation of the first part of the optical device 102 including the structures 106 (FIG. 2) for example as described above, in particular after removal of the mold 110 of FIG. 8 at the end of the evaporation of the solvent from the first deposited solution 109. Furthermore, the step of forming the optical device 102 includes, after evaporation of the solvent from the first deposited solution 109:
a step of depositing a second solution 112, for example by depositing a drop of this second solution 112, on the structures 106 (FIG. 12) formed on the emission face 103 of the light-emitting diode 101 and in particular on the substrate 108, in order to form a second part of the optical device 102, the second solution 112 including a solvent (for example the same as that of the first solution 109) and a second part of the transparent particles 104 of the optical device 102 to be formed,
a step of evaporating the solvent from the second deposited solution 112 to form the second part of the optical device 102 corresponding to the optical element 105 described above.
   The first solution 109, or the second solution 112, includes the conversion particles 107. The step of evaporating the solvent from the second deposited solution 112 can be carried out at the same temperatures as described in relation to the evaporation of zo the solvent of the first deposited solution 109, from which the same advantage results. According to this second embodiment, it was possible to form the optical device 102 of a suitable shape without damaging the light-emitting diode 101. Here an advantage is that the second deposited solution 112 can directly have a shape representative of the desired shape of the optical device 102 in adjusting the viscosity of the second solution 112.

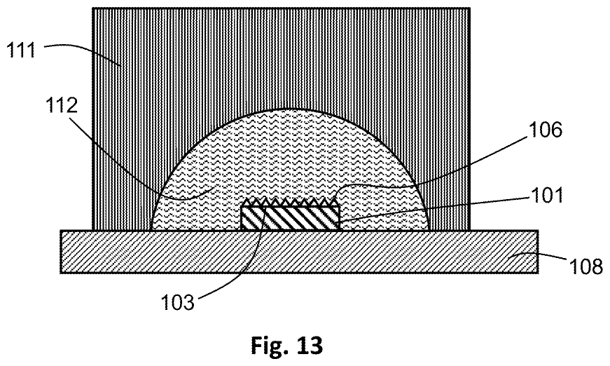

Alternatively to depositing the second solution 112 in the form of a drop, it is possible to deposit the second solution 112 in any shape, then to shape it using a suitable mold 111 (FIG. 13) whose position is maintained during the evaporation of the solvent from the second deposited solution 112. However, before depositing the second solution 112, an annealing is carried out here to stabilize the structures 106 arranged on the emission face 103 of the light-emitting diode 101 so as to prevent damage to these structures 106 during the molding of the second part of the optical device 102.

According to yet another alternative to depositing the second solution in the form of a drop, it is possible, after evaporation of the solvent from the first deposited solution 109, to deposit a protective layer of bulk material, for example of aluminum nitride, to seal the structures 106 and protect them before depositing the second solution 112 and giving it a desired shape by molding.

According to a variant of the second embodiment of the manufacturing method, the first solution is deposited on the structures previously formed by depositing the second solution on the emission face of the light-emitting diode. In this variant, the second solution includes the conversion particles, and the second solution is devoid of transparent particles which are then all contained in the first solution. Before depositing the first solution, the second solution deposited on the emission face is notably shaped and its solvent evaporated to form the structures. Then, after depositing the first solution on the structures, the solvent of the first deposited solution can be evaporated to form the optical element, if necessary while a corresponding mold ensures the maintenance of the shaping of the first deposited solution according to the desired shape of the optical element.

It results from what has been described above that the production of the optical device 102 of the optoelectronic device 100 can, where appropriate, be implemented without having to deposit material using microelectronic techniques at a high temperature which could have the consequence of damaging the light-emitting diode 101.

It results from what has been described above that, in the context of shaping the deposited solution 109, the step of forming the optical device 102 may include a step of shaping the solution deposited in order to form at least a part of the optical device 102. In this case, the step of evaporating the solvent from the deposited solution 109 is carried out for the deposited and shaped solution 109, that is to say that the shape imparted to the solution by the shaping step is maintained throughout the step of evaporating the solvent of solution 109.

It was previously mentioned that the optical device 102 could also cover the lateral flanks of the light-emitting diode 101. Thus, an advantage of the used optical device 102 is to fill in the trenches delimiting the light-emitting diode and, where appropriate, to fill in spaces between light-emitting diodes when the optoelectronic device includes several which can in particular share the same optical device 102. This also allows to facilitate the extraction of photons from the lateral flanks of the light-emitting diode, and where appropriate, to allow their conversion at least a part.

Preferably, when the optical device 102 includes the optical element 105, the light-emitting diode 101 is small compared to the optical element 105 and is located in particular at the center of the base of the optical element 105 which surmounts the light-emitting diode 101 and in particular the substrate 108.

Anything that applies to optoelectronic device 100 may apply to its manufacturing method and vice versa.

The optoelectronic device 100 has industrial application in the field of manufacturing such an optoelectronic device, as well as in any application requiring ultraviolet lighting.

1. An optoelectronic device including:
a light-emitting diode configured to emit an electromagnetic radiation at an emission wavelength of the light-emitting diode comprised in the ultraviolet,
an optical device configured to extract photons generated by the light-emitting diode,
said optical device being arranged on an emission face of the light-emitting diode, said optical device including:
transparent particles at the emission wavelength of the light-emitting diode,
conversion particles,
wherein the conversion particles are configured so as to emit, by converting a part of the electromagnetic radiation emitted by the light-emitting diode, an electromagnetic radiation at an emission wavelength of the conversion particles comprised in the ultraviolet and strictly greater than the emission wavelength of the light-emitting diode.

2. The optoelectronic device according to claim 1, wherein the emission wavelength of the light-emitting diode is selected in the ultraviolet C, and in that the emission wavelength of the conversion particles is selected in the ultraviolet A.

3. The optoelectronic device according to claim 1, wherein the optical device has the shape of a dome, a cone or a pyramid.

4. The optoelectronic device according to claim 1, wherein the optical device includes a plurality of structures, these structures being arranged on the emission face of the light-emitting diode, each of the structures including a part of the transparent particles.

5. The optoelectronic device according to claim 4, wherein the structures each include a part of the conversion particles.

6. The optoelectronic device according to claim 1, wherein the optical device includes an optical element and structures, the structures being arranged on the emission face of the light-emitting diode, the optical element being arranged on the structures, and in that:
the optical element includes the conversion particles, the transparent particles being distributed such that:
the structures each include transparent particles, and
the optical element includes transparent particles, or
the conversion particles are distributed so that the structures each include conversion particles, the transparent particles being distributed so that the optical element includes transparent particles and that the structures include transparent particles, or
the structures include only the conversion particles and the optical element includes the transparent particles.

7. The optoelectronic device according to claim 1, wherein the transparent particles are each made of aluminum nitride.

8. The optoelectronic device according to claim 1, wherein the conversion particles are each made of gallium nitride.

9. The optoelectronic device according to claim 1, wherein each of the transparent particles has a size strictly less than λ/(2×n), with n the optical index of the material of the transparent particles, and λ the emission wavelength of the light-emitting diode.

10. The optoelectronic device according to claim 1, wherein each of the conversion particles has a size strictly less than λ/(2×n₁), with n₁ the optical index of the material of the conversion particles, and λ the emission wavelength of the light-emitting diode.

11. The optoelectronic device according to claim 1, wherein the cohesion of the optical device is provided by van der Waals bonds.

12. A method for manufacturing an optoelectronic device according to claim 1, wherein it includes the following steps:
a step of supplying the light-emitting diode,
a step of forming the optical device including:
a step of depositing a solution at least above the emission face of the light-emitting diode, said solution including a solvent and at least a part of the transparent particles,
a step of evaporating the solvent from the deposited solution to form at least a part of the optical device.

13. The manufacturing method according to claim 1, wherein:
the solution deposited at least above the emission face is a first solution including a first part of the transparent particles of the optical device to be formed,
the step of forming the optical device includes a step of shaping the first deposited solution,
it results from the step of shaping the first deposited solution and the step of evaporating the solvent from the first deposited solution, a formation of a first part of the optical device including structures,
the step of forming the optical device includes:
a step of depositing a second solution on the structures in order to form a second part of the optical device, the second solution including a solvent and a second part of the transparent particles of the optical device to be formed,
a step of evaporating the solvent from the second deposited solution to form the second part of the optical device,
the first solution, or the second solution, includes conversion particles configured so as to emit, by converting a part of the electromagnetic radiation emitted by the light-emitting diode, an electromagnetic radiation at an emission wavelength of the conversion particles comprised in the ultraviolet and strictly greater than the emission wavelength of the light-emitting diode.
